(19) United States
(12) Patent Application Publication
(10) Pub. No.: US 2005/0182387 A1
(43) Pub. Date: Aug. 18, 2005

(54) Peel-away catheter shaft

(75) Inventor: William Earl Webler, Escondido, CA (US)

Correspondence Address: CRAWFORD MAUNU PLLC, 1270 NORTHLAND DRIVE, SUITE 390, ST. PAUL, MN, 55120, US

(73) Assignee: Cardiac Pacemakers, Inc.

(21) Appl. No.: 10/778,270

(22) Filed: Feb. 13, 2004

(51) Int. Cl. A61M 25/00

(52) U.S. Cl. 604/527; 604/160; 604/164.05

(57) ABSTRACT

A peel-away shaft for a guiding catheter includes a shaft wall defining an open lumen of the catheter shaft. A peel-away region is defined over at least a partial length of the catheter shaft. A braid substantially encompasses the open lumen. The braid includes a region of discontinuity extending along the peel-away region. The region of discontinuity of the braid reduces the tear resistance of the shaft wall along the peel-away region.

### FIELD OF THE INVENTION

The invention relates generally to catheter systems, and, more particularly, to catheters having peel-away shafts.

### BACKGROUND

Catheters are commonly used in medical procedures for providing access to a patient's internal anatomy. Catheters can be used to assist in a wide range of procedures, including ablation, drug treatments, measurement, mapping, and device implantation. In many procedures, catheters are used to locate and cannulate vessels in support of the procedure. Cannulating heart vessels may require navigating a small diameter, flexible guide through the body into a destination vessel, such as those in the heart. Once the destination vessel is reached, the catheter can act as a conduit for insertion of payloads into the vessel. Catheters used in this manner are commonly referred to as guide catheters.

Guide catheters are typically specially adapted for a particular type of procedure. For example, guide catheters adapted for implanting cardiac devices (e.g., pacing leads) include an open lumen dimensioned to receive the cardiac device. These cardiac guide catheters may also be designed with the appropriate shape, length, and flexibility to be effective for maneuvering in the access path used in the procedure.

Although properly placing the guide catheter is challenging, removing the catheter has its own set of unique problems. After implanting a cardiac pacing lead, for example, the guide catheter must be removed from around the lead. This is done by proximally retracting the catheter until it is completely withdrawn from around the lead. During withdrawal, there may be considerable jostling of the catheter and lead while the catheter is being manipulated at the proximal end, which may result in the lead being dislodged or damaged. In other cases, the lead may have a proximal part that will not fit through the guide catheter, and the catheter must be cut away during withdrawal, which slows down the procedure and risks dislodging the lead. Therefore, there is a need to reduce the potential of lead perturbation during guide catheter withdrawal.

### SUMMARY OF THE INVENTION

The present disclosure describes a peel-away shaft for a guiding catheter. In one embodiment, a catheter shaft includes a shaft wall defining an open lumen of the catheter shaft. A peel-away region is defined over at least a partial length of the catheter shaft. A braid substantially encompasses the open lumen. The braid includes a region of discontinuity extending along the peel-away region. The region of discontinuity of the braid reduces the tear resistance of the shaft wall along the peel-away region.

The region of discontinuity may include a gap extending along the peel-away region and/or a bead of polymeric material disposed within the region of discontinuity of the braid. The polymeric material may include a visually distinct color. In one configuration, the braid further includes a second region of discontinuity extending along a second peel-away region defined over at least a partial length of the catheter shaft. In another arrangement, the peel-away region may follow a substantially longitudinal path along the catheter shaft, and/or follow a substantially helical path along the catheter shaft. An outer jacket may substantially encompass the braid. The outer jacket may include a peel-away feature extending along the peel-away region. The peel-away feature of the outer jacket reduces the tear resistance of the outer jacket along the peel-away region. The catheter shaft may include an inner liner disposed along the open lumen.

In another embodiment of the present invention, a method of implanting a device in a destination vessel involves implanting the device through an open lumen of a catheter shaft that cannulates the destination vessel. The catheter shaft is separated at a proximal portion of a peel-away region of the catheter shaft. The peel-away region is defined over at least a partial length of the catheter shaft. The catheter shaft is withdrawn while continuing to separate the catheter along a discontinuity of a braid of the catheter shaft. The discontinuity of the braid reduces tear resistance of the catheter shaft along the peel-away region. The catheter shaft is then removed from the device.

In another embodiment of the present invention, a method of making a catheter shaft involves providing a form that defines an open lumen of the catheter shaft. A braid is provided as a flattened sheet and wrapped around the form so that there is a discontinuity in the braid over at least a portion of the length of the catheter shaft. The braid is joined together along the discontinuity of the braid.

The above summary is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

FIG. 1 is a side view of a peel-away catheter shaft according to various embodiments of the present invention;

FIG. 2 is a sectional perspective view of shaft construction according to various embodiments of the present invention;

FIG. 3 is a cross-sectional view of the catheter shaft of FIG. 2 according to various embodiments of the present invention;

FIG. 4 is a cross-sectional view of a catheter shaft having dual peel-away features according to various embodiments of the present invention;

FIG. 5 is a perspective view of a catheter shaft having a helical peel-away feature according to various embodiments of the present invention;

FIG. 6 is a cross sectional view illustrating forming of a braid sheet according to various embodiments of the present invention;

FIGS. 7A-D illustrate steps in making a catheter shaft according to various embodiments of the present invention;

FIG. 8 is a cross sectional view illustrating an alternate peel-away braid configuration according to various embodiments of the present invention; and

FIG. 9 is a cross sectional view illustrating an overlapping peel-away braid configuration according to various embodiments of the present invention

While the invention is amenable to various modifications and alternative forms, specifics thereof have been shown by way of example in the drawings and will be described in detail herein. It is to be understood, however, that the intention is not to limit the invention to the particular embodiments described. On the contrary, the invention is intended to cover all modifications, equivalents, and alternatives falling within the scope of the invention as defined by the appended claims.

### DETAILED DESCRIPTION

In the following description of the illustrated embodiments, references are made to the accompanying drawings which form a part hereof, and in which is shown by way of illustration, various embodiments in which the invention may be practiced. It is to be understood that other embodiments may be utilized, and structural and functional changes may be made without departing from the scope of the present invention.

In broad and general terms, the present invention relates to a catheter shaft that provides the benefits of a braided shaft wall construction while allowing the shaft to be easily peeled away when being removed from the body following a medical procedure. This shaft construction may be useful in many applications, particularly where the catheter is used to guide an implantable payload that is intended to be left in place after the procedure is complete. This type of payload may include implantable leads, as well as measuring or monitoring apparatus. The present disclosure describes examples of catheters used for pacing lead implantation. However, it will be appreciated by those skilled in the art that the concepts described may be applied to other applications where it is desired to withdraw a catheter without disturbing an in-place apparatus.

Typically, operations such as cardiac lead implantation can be performed by advancing the lead through a guide catheter that has been inserted through the vasculature. Even though guiding and placing the catheter in the desired heart location via a transvenous approach can sometimes be difficult, this procedure is generally safer than performing surgery on or near the heart to implant the object.

Withdrawing a guide catheter that contains a recently implanted lead may be a delicate operation. It will be appreciated that having the catheter shaft peel-away during retraction is of great benefit. Peeling away the catheter allows the catheter to be removed without using an extra-long lead or some method of holding the lead in place until the distal tip of the catheter is pulled from around the lead. A peel-away catheter is also useful where the proximal part of the lead is enlarged and will not fit through the guide catheter lumen.

A peel-away catheter shaft should preferably have mechanical characteristics similar to a non-peel-away guiding catheter shaft. These physical characteristics of the shaft include bending flexibility, kink resistance, and good torsional and longitudinal rigidity. The peel-away feature should allow the shaft, once an initial break is made in a proximal part of the shaft wall, to be relatively easily separated while the shaft is being withdrawn. This separation may be done by hand or with the assistance of a cutting tool. To prevent injury to the physician or the patient, the separated portions of the shaft should not present any sharp ends, such as those presented by the exposed ends of cut braiding wires.

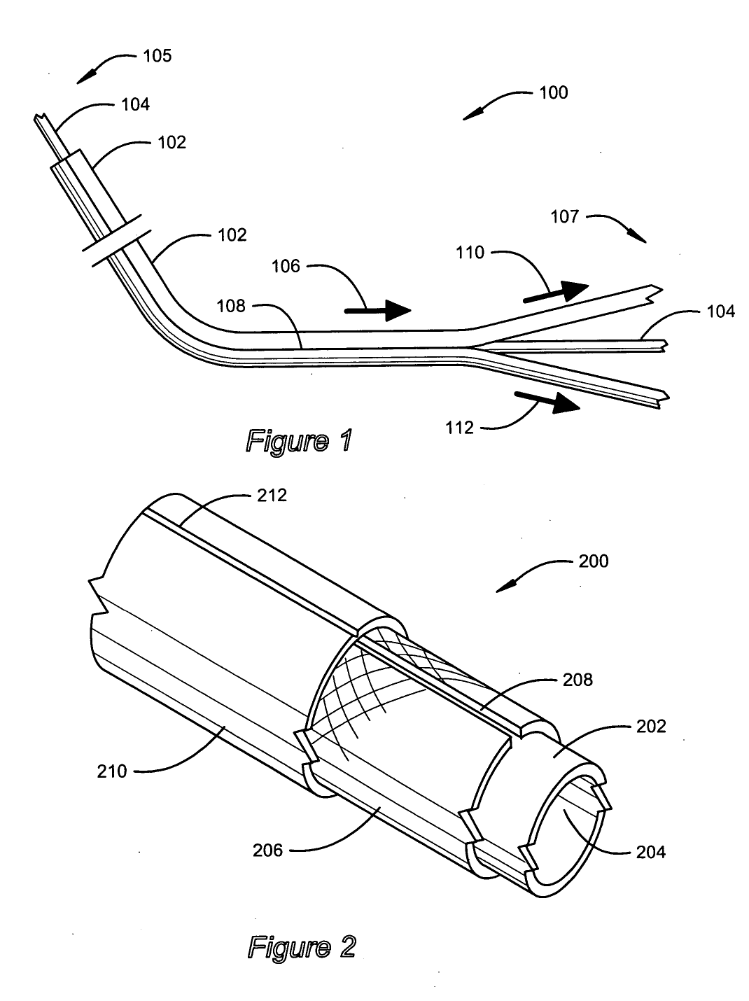

With reference now to FIG. 1, a guiding catheter 100 is illustrated according to embodiments of the present invention. The guiding catheter 100 includes an elongated, flexible shaft 102 that can be introduced in anatomical passageways to support medical procedures. The medical procedures may include insertion of medical devices into the heart, as well as delivery of drugs, sensors, pacing leads, defibrillation leads, or other diagnostic objects into target vessels of the body.

The catheter shaft 102 is typically a cylindrical tube, having an internal lumen for the passage of a payload 104. It will be appreciated that the concepts described herein may also apply to catheters formed with a variety of cross sectional shapes. Similarly, the concepts may be applied to shafts with additional internal features (e.g., extra lumens, steering apparatus), and shafts having features and dimensions that vary across the catheter's length. Generally, the catheter shaft 102 is used to place a distal end 105 of the payload 104 in an anatomical location such as a heart cavity. Once the payload 104 is implanted, the catheter shaft 102 is withdrawn in a proximal direction, as indicated by the arrow 106.

To prevent displacing or jarring the payload 104 during catheter withdrawal, the catheter shaft 102 includes a peel-away feature 108 that allows a proximal end 107 of the shaft 102 to be separated during withdrawal. A mechanical attachment, such as a separable handle (not shown) may help initiate splitting of the catheter shaft 102 at the catheter's proximal end 107. Thereafter, the catheter shaft 102 is separated by pulling the shaft's proximal end 107 as indicated by the arrows 110, 112. This pulling may be accompanied by cutting at or near the peel-away feature 108 using a tool.

The peel-away feature 108 may include a longitudinal pre-stress along the length of the catheter shaft 102. Such a pre-stress line may be suitable for a catheter having a simple, single-material shaft construction. However, most modern catheters are formed by building up multiple layers of materials. Typically, a shaft formed from a single material cannot provide a combination of desirable shaft characteristics, such as flexibility, kink-resistance, and torsional rigidity for ease of manipulation and lumen lubricity for facilitating passage of the payload 104. The formation of an effective peel-away feature 108 in a modern, multi-layer catheter shaft poses challenges due to the complex shaft construction and performance requirements of the finished product.

In reference now to FIG. 2, a section of a multi-layer, peel-away catheter shaft 200 is illustrated according to embodiments of the present invention. The shaft 200 includes an inner liner 202 that may include one or more open lumens 204 adapted for the passage of a catheter payload. The inner liner 202 may be fabricated from a polymer tube, usually a thin fluorocarbon or other low friction tube. To further reduce friction within the tube, the liner 202 may include a lubricious coating (e.g., a hydro-coat, a hydrophilic coating, or a silicone oil coating) along the inner surface that defines the lumen 204. To provide an improved adhesive surface to adhere to the impregnation material of braid 206, the outer surface of liner 202 may be etched (e.g., chemical or plasma etching). An etching process may also be used on the inside surface of the liner 202 or guide shaft to aid in the adherence of a lubricious coating.

Surrounding the inner liner 202 is a braid 206. A guide catheter shaft 200 often includes a braid 206 embedded in the shaft walls to provide desirable physical properties to the catheter (e.g., one or more of torsional stiffness, pushing stiffness, and kink resistance). The braid 206 may be formed from round or flat metallic wires (e.g., stainless steel) or from strands of fibrous material (e.g., Vectran®, Kevlar®), for example. Metallic braids may be impregnated with a material that allows it to easily bond with polymeric materials used to form other parts of the catheter shaft. This impregnation material (e.g., Pebax® or Pebax®-nylon blends) usually provides the bulk of the flexural characteristics of the produced tube/guide. As such, the composition of the impregnation material may be varied along the length of the tube/guide to provide differing flexural characteristics to aid in guide positioning. Typically, a guide catheter is made to be more flexible near its distal end.

Generally, a tube-like braid that entirely surrounds the catheter shaft may impart desirable physical properties to a shaft, but is difficult to peel or cut away during shaft extraction. The illustrated braid 206, however, is formed such that it does not completely surround the inner liner 202. Instead, the braid 206 is formed with a discontinuity, e.g., a gap 208. The gap 208 extends along at least a partial length of the shaft 200 and provides a convenient separation point for peeling away the catheter shaft 200. The gap 208 may be contiguous along the catheter shaft 200, or the gap 208 may have interruptions along the catheter shaft 200, such that the gap 208 resembles a perforated line.

By forming the braid 206 with a gap 208, the shaft 200 can still retain desired physical properties, yet be made easier to peel or cut away. Although one gap 208 is shown in the braid 206, it will be appreciated that any appropriate number of similar gaps may be included in the braid 206. The shaft 200 may also contain additional braids either interior to or exterior to the gapped braid 206. For example, the inner liner 202 may be formed using a braided tube. Any additional braids included in the shaft 200 may also contain a gap, although a gap may not be needed if the additional braids are formed using dimensions and/or materials that are relatively easy to separate during use. Also, such an additional braid should be separable without adverse effects such as leaving exposed sharp ends after separation.

The gap 208 may be filled with a bead of material during lay-up of the braid 206 on the shaft 200. The material may be a polymer material (e.g., nylon, Pebax®) that is melted or molded into place during shaft construction. The material used to fill in the gap 208 is typically miscible with the material in which the braid 206 is impregnated so as to structurally join the ends of the braid 206. The gap-filling material may have a color that contrasts with the color of the impregnation material of braid 206 for aiding alignment during shaft construction, as well as for providing a guide for benefit of the end user.

In other configurations, the gap 208 may be bridged by weaving a joining member or members (e.g., wire, thread) between the edges of the braid 206 adjacent to the gap 208. Such a joining member would preferably be formed of material that has a high melting point and is easily separated or cut (e.g., Vectran®, Kevlar®). Use of a joining member may result in the gap 208 becoming very small, or even result in the gap 208 being closed altogether. The joining member could be used alone or in combination with a bead of gap-sealing material.

In some configurations of the catheter shaft 200, the braid 206 may be impregnated with a miscible material that is molded into a smooth outer surface of the shaft 200 suitable for medical use. In other configurations, an outer member, or jacket, may be provided to ensure a smooth shaft outer surface. In FIG. 2, an outer jacket 210 is shown surrounding the braid 206. The outer jacket 210 may be formed from a smooth, abrasion resistant polymer. The outer jacket 210 may be formed of the same materials used to impregnate the braid 206. The outer jacket 210 can be formed of a clear or translucent material if it is desired that the braid gap 208 be made visible to the end user. The outer jacket 210 may include one or more peel-away features 212 that are co-located with the braid gap(s) 208. It will be appreciated that the peel-away feature 212 may be provided as an indicator of location of the underlying braid gap 208, as well as providing a pre-stress for ease of separating the outer jacket 210. In the latter case, the peel-away feature 212 need not be formed adjacent to the braid gap 208, assuming it is acceptable to remove the outer jacket 210 separately from the braid 206.

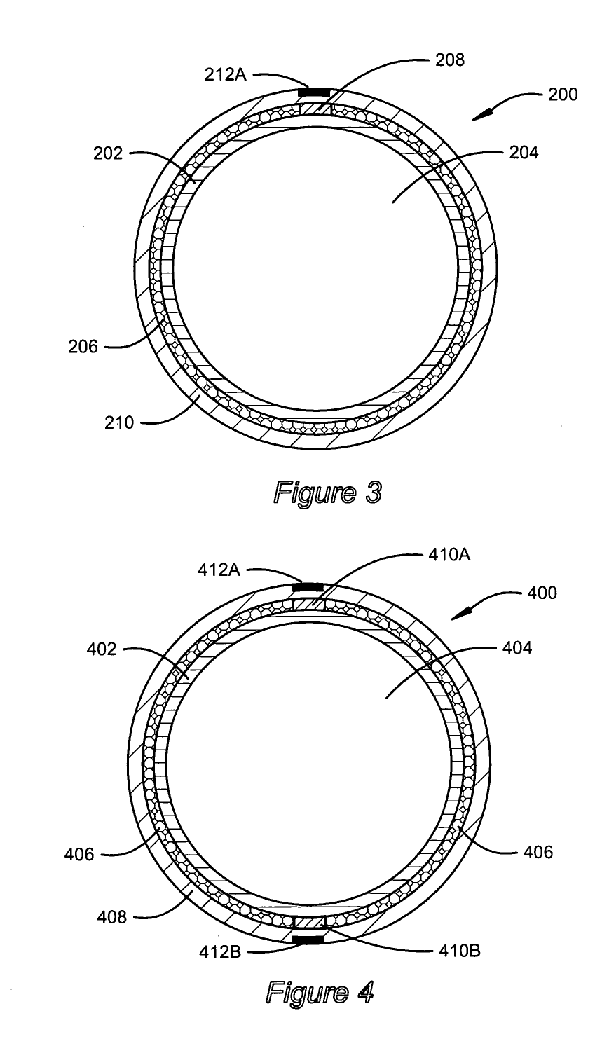

The peel-away feature 212 may include a pre-stress line (e.g., notch) that eases removal of the outer jacket 210. A similar pre-stress line may be included in the inner liner 202. In other arrangements, the peel-away feature 212 may be a molded-in feature of the outer jacket 210, such as a different color material and/or an embedded fiber or wire. An embedded fiber or wire may be installed such that the pulling/removal of the fiber creates the pre-stress line or a deeper notch or gap and thus facilitates the peel-away action. An alternate peel-away feature 212A is shown in FIG. 3, which shows a cross-section of the shaft section 200 of FIG. 2 according to embodiments of the present invention. The alternate peel-away feature in FIG. 3 includes a different color material embedded in the outer jacket 210.

Although the shaft construction shown in FIGS. 2 and 3 includes a single braid gap 208, it will be appreciated that multiple braid gaps may also be used. In FIG. 4, a cross-section of multiple-braid-gap catheter shaft 400 is illustrated according to embodiments of the present invention. The shaft 400 includes an inner liner 402, lumen 404, braid 406, and outer jacket 408 similar to previously described configurations. The braid 406 in this arrangement includes two gaps, 410A and 410B. The braid 406 may be formed from two completely detached portions to accomplish the construction shown. Also shown in FIG. 4 are two peel-away features 412A and 412B on the outer jacket 408.

The illustrated gaps 410A, 410B and peel-away features 412A, 412B may be disposed at a substantially constant radial location on the shaft's outer surface. This results in a substantially longitudinal pattern that extends across the length of the catheter. This longitudinal pattern can be seen, for example, in the peel-away feature 108 of FIG. 1. It will be appreciated that other peel-away patterns may be used.

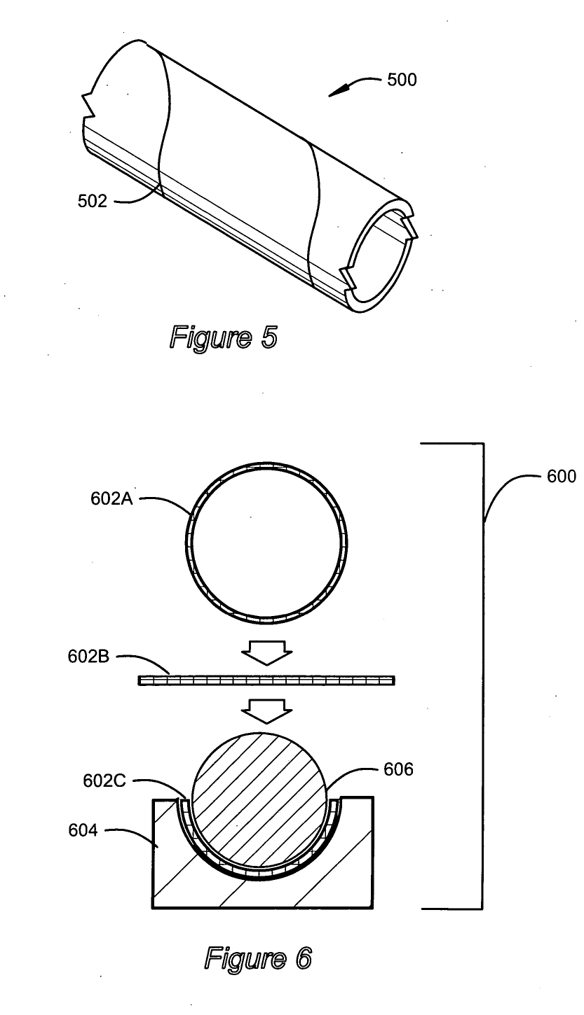

FIG. 5, for example, illustrates an alternate layout of a peel-away catheter shaft 500 according to embodiments of the present invention. The catheter shaft 500 includes a helical peel-away pattern 502. Both the braid and outer jacket may include peel-away features having the helical peel-away pattern 502. The helical pattern 502 may be less prone to accidental splitting of the shaft due to normal use.

A multi-layer shaft with an impregnated braid may be formed using different techniques depending on the end result required. Typically, the braid is woven on a cylindrical form known as a blocker. The blocker is used to maintain the tubular shape during construction steps and is made of materials that will release from the constructed tube/guide and be removed when appropriate. Usually an inner liner may be first placed over the blocker, and the braid woven on the outside of the inner liner. In another arrangement, the braid may be fabricated on the blocker without a liner and then removed for use as-is. The braid is typically impregnated with a material that is compatible with the material used to form the adjacent layers of the guide. Typically, tubes of the desired impregnation material are placed over the braid and then placed inside a shrink tube (e.g. FEP shrink tubing). When exposed to heat, the impregnation material is melted and forced by the shrinking heat shrink into the gaps of the braid to form the impregnation. Once cooled, the heat shrink and the blocker are removed. The result is a tube-like shaped braid that is suitable for accessing the anatomy.

Techniques similar to those described above may also be used to form a braid having a gap. Referring again to FIG. 3, the braid 206 may be formed on a blocker as described above, then the braid 206 can be cut down the side to form the desired gap 208. However, cutting the braid 206 may result in protruding sharp wire ends, and a cut braid has a tendency to unravel. An alternate technique of forming a gapped braid according to embodiments of the present invention is shown in FIG. 6.

In FIG. 6, various stages 600 are shown for forming a braid that may include a gap devoid of sharp edges or other problems inherent in cutting the braid. The braid is formed and impregnated as a tube-like shape 602A using a blocker or equivalent. The tubular shape 602A is flattened into a sheet 602B using a roller or press which may be accompanied by the application of heat, then cooling to aid in reshaping the impregnation material. The sheet 602B may be then be used to form a gapped braid 602C that conforms to the shape of the catheter shaft. The gapped braid 602C may be formed using a mold 604 and/or a mandrel 606 and may be accompanied by the application of heat, then cooling to aid in reshaping the impregnation material. The gapped braid 602C may also be given its final shape during lay-up with other components of the catheter shaft.

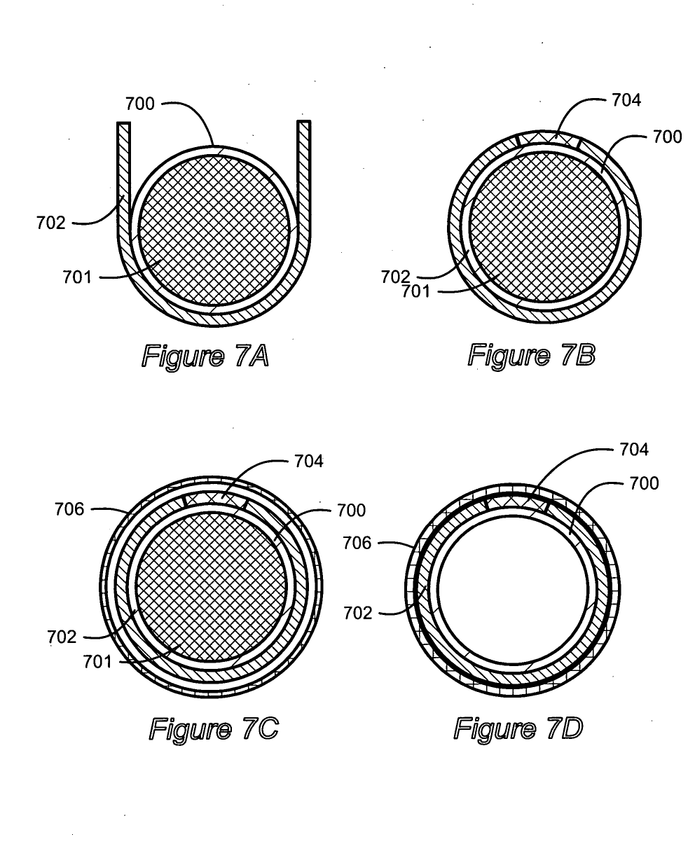

An illustrative shaft lay-up procedure according to embodiments of the present invention is illustrated in FIGS. 7A-D. In FIG. 7A, a braid 702 is folded over a liner 700. The liner 700 may be supported during this procedure by a blocker 701. The braid 702 may be a substantially flattened braid formed as described in relation to FIG. 6. The braid 702 is dimensioned to leave a gap 704 as seen in FIG. 7B. The gap 704 can be filled with a bead of material to join the edges of the braid 702 or may rely on the flowing of the impregnation material in the next step of the process. The assembly may then be inserted into a heat shrink tube, exposed to heat and then cooled. This melts the impregnation material (and the bead of material, if present) and causes it to adhere to the outside of liner 700. If and when the heat shrink is removed (heat shrink tubes that provide the required forming pressures are often too stiff to remain a part of the guide), an assembly as shown in FIG. 7B remains. In some configurations, all that is necessary at this point to produce a usable guide is to remove the blocker 701. The jacket is formed by the melted impregnation material.

In other configurations, the heat shrinking process and/or the residual stresses in the braid 702, cause a portion of the braid 702 to be visible through the impregnation material. Visible exposure of a portion of the braid 702 is considered a safety hazard. When the guide is flexed and/or passed through other devices during use, it is possible for a braid 702 wire loop to come free of the impregnation. This wire loop may damage the inside of the veins or arteries as the guide is inserted into the body. To solve this problem an outer jacket 706 may be incorporated. The assembled liner 700 and braid 702 are placed within an outer jacket 706 as shown in FIG. 7C. The outer jacket 706 in this example is a heat shrink tube formed from a thin flexible material (e.g., 0.00025 inch wall polyester heat shrink tube). This heat shrinkable material preferably has a shrinking temperature less than the melting temperature of the gap filler material or impregnation material to avoid detaching the braid 702 from liner 700 or deforming the braid 702 during the shrinking process. Application of heat to the outer jacket 706 causes it to tightly shrink around the braid 702 as seen in FIG. 7D. In some configurations, the thin flexible outer jacket 706 may be applied to sufficiently bind the braid 702 so that no material is needed to fill the gap 704 as shown in FIG. 7B and/or no previous heat shrink step is required.

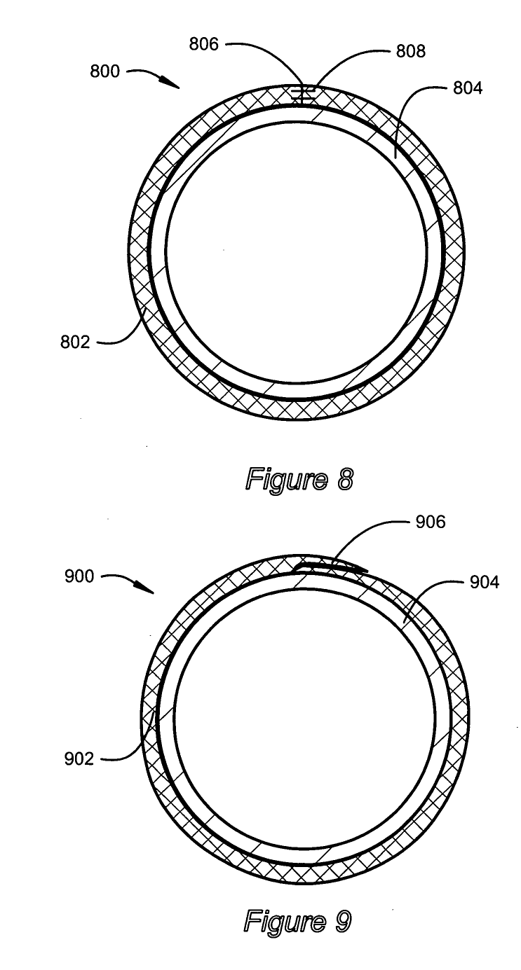

The procedures described above for forming a gapped braid arrangement may also be used to form other splittable braid configurations. One configuration of a shaft 800 splittable braid in accordance with embodiments of the present invention is shown in FIG. 8. A split braid 802 is laid around an inner liner 804 so that the edges of the braid 802 are substantially in contact at a joint area 806 that serves as a discontinuity of the braid 802. A binding member 808 can be threaded or sewn through the braid ends to join the braid together over the inner liner 804. The binding member can be any type of thread or wire, typically chosen to break or cut easier than the braid 802. It will be appreciated that a polymeric material that is miscible with an impregnable braid 802 may also be used to join the edges at the joint area 806. In other configurations, the edges may be joined by soldering or welding.

FIG. 9 shows an alternate configuration of a shaft 900 with a splittable braid in accordance with various embodiments of the present invention. The split braid 902 is again laid around an inner liner 904. In this configuration, a discontinuity is formed by the ends of the braid 902 being overlaid at the joint area 906. The overlapped portions of the braid 902 may be formed thinner than the rest of the braid 902 to prevent excessive bulging at the joint area 906. The braid 902 may be attached at the joint area 906 by any manner described herein, such as by injecting a bonding material between the overlaid portions, by melting the impregnation material together, and/or by using a binding member.

It will, of course, be understood that various modifications and additions can be made to the embodiments discussed hereinabove without departing from the scope of the present invention. Accordingly, the scope of the present invention should not be limited by the particular embodiments described above, but should be defined only by the claims set forth below and equivalents thereof.

1. A catheter shaft, comprising:
a shaft wall defining an open lumen of the catheter shaft;
a peel-away region defined over at least a partial length of the catheter shaft; and
a braid substantially encompassing the open lumen, the braid including a region of discontinuity extending along the peel-away region, wherein the region of discontinuity of the braid reduces the tear resistance of the shaft wall along the peel-away region.

2. The catheter shaft of claim 1, further comprising an outer jacket substantially encompassing the braid.

3. The catheter shaft of claim 2, wherein the outer jacket comprises a peel-away feature extending along the peel-away region, the peel-away feature reducing the tear resistance of the outer jacket along the peel-away region.

4. The catheter shaft of claim 1, further comprising a liner disposed along the open lumen, the liner defining an inner surface of the open lumen.

5. The catheter shaft of claim 1, wherein the region of discontinuity comprises a gap extending along the peel-away region.

6. The catheter shaft of claim 1, further comprising a bead of polymeric material disposed within the region of discontinuity of the braid.

7. The catheter shaft of claim 6, wherein the polymeric material includes a visually distinct color.

8. The catheter shaft of claim 1, further comprising a second peel-away region defined over at least a partial length of the catheter shaft, the braid further comprising a second region of discontinuity extending along the second peel-away region.

9. The catheter shaft of claim 1, wherein the peel-away region extends along a substantially longitudinal path along the catheter shaft.

10. The catheter shaft of claim 1, wherein the peel-away region extends along substantially helical path along the catheter shaft.

11. The catheter shaft of claim 1, wherein the braid member comprises a stainless steel braid.

12. The catheter shaft of claim 1, wherein the region of discontinuity is interrupted along the peel-away region.

13. A method of implanting a device in a destination vessel, comprising:
implanting the device through an open lumen of a catheter shaft that cannulates the destination vessel;
separating the catheter shaft at a proximal portion of a peel-away region of the catheter shaft, the peel-away region defined over at least a partial length of the catheter shaft;
withdrawing the catheter shaft while continuing to separate the catheter along a discontinuity of a braid of the catheter shaft, the discontinuity of the braid reducing tear resistance of the catheter shaft along the peel-away region; and
removing the catheter shaft from the device.

14. The method of claim 13, wherein the peel-away region extends along a substantially longitudinal path along the catheter shaft.

15. The method of claim 13, wherein the peel-away region extends along a substantially helical path along the catheter shaft.

16. The method of claim 13, wherein withdrawing the catheter shaft further comprises separating the catheter along a second discontinuity of the braid of the catheter shaft, the second discontinuity of the braid reducing tear resistance of the catheter shaft along a second peel-away region defined over at least a partial length of the catheter shaft.

17. The method of claim 13, wherein the discontinuity is uninterrupted along the peel-away region.

18. A method of making a catheter shaft, comprising:
providing a form that defines an open lumen of the catheter shaft;
providing a braid configured as a flattened sheet;
wrapping the braid around the form so that there is a discontinuity in the braid over at least a portion of the length of the catheter shaft; and
joining together the braid along the discontinuity of the braid.

19. The method of claim 18, wherein the discontinuity comprises a gap in the braid.

20. The method of claim 18, wherein joining together the braid comprises depositing a bead of polymeric sealing material along the discontinuity of the braid.

21. The method of claim 18, wherein joining together the braid comprises heat-shrinking a tubular member situated around the braid.

22. The method of claim 18, further comprising flattening a substantially tubular braid to form the braid configured as the flattened sheet.

23. The method of claim 18, further comprising forming the flattened braid into a curved shape that substantially conforms to a shape of the tubular inner liner.

24. The method of claim 18, further comprising:
providing an inner liner of the catheter shaft; and
situating the inner liner to define an inner surface of the open lumen of the catheter shaft.

25. The method of claim 18, further comprising situating an outer jacket of the catheter shaft over the braid.

26. The method of claim 18, further comprising forming a peel-away feature in the outer jacket substantially aligned with the discontinuity of the braid, the peel-away feature reducing the tear resistance of the outer jacket along the discontinuity of the braid.

27. The method of claim 18, wherein the region of discontinuity is uninterrupted along the peel-away region.

28. A catheter shaft, comprising:
a shaft wall defining an open lumen of the catheter shaft;
reinforcement means for providing kink-resistance of the catheter shaft, the reinforcement means substantially encompassing the open lumen; and
means for facilitating separation of the reinforcement means along a peel-away region defined over at least a partial length of the catheter shaft.

29. The catheter shaft of claim 28, further comprising jacketing means for providing a smooth covering over the separation means.

30. The catheter shaft of claim 29, further comprising means for facilitating separation of the jacketing means along the peel-away region.

31. The catheter shaft of claim 28, further comprising means for providing a lubricious inner surface of the open lumen.
